SEQUENCE LISTING (13 sequences)

SEQ ID NO: 1 - DNA, 5013 bp, Homo sapiens
atgagcgcccggaccgcccccaggccgcaggtgctcctgctgccgctcctgctggtgctcctggcggcggcgcccgcagccagcaagggttgtgtctgtaaagacaaaggccagtgcttctgtgacggggccaaaggggagaagggggagaagggctttcctggaccccccggttctcctggccagaaaggattcacaggtcctgaaggcttgcctggaccgcagggacccaagggctttccaggacttccaggactcacgggttccaaaggtgtaaggggaataagtggattgccaggattttctggttctcctggacttccaggcaccccaggcaataccgggccttacggacttgtcggtgtaccaggatgcagtggttctaagggtgagcaggggtttccaggactcccagggacactgggctacccagggatcccgggtgctgctggtttgaaaggacaaaagggtgctcctgctaaagaagaagatatagaacttgatgcaaaaggcgaccccgggttgccaggggctccaggaccccagggtttgccaggccctccaggttttcctgggcctgttggcccacctggtcctccgggattctttggctttccaggagccatgggacctagaggacctaagggtcacatgggtgaaagagtgataggacataaaggagagcggggtgtgaaagggttaacaggacccccgggaccaccaggaacagttattgtgaccctaactggcccagataacagaacggacctcaagggggaaaagggagacaagggagcaatgggcgagcctggacctcctggaccctcaggactgcctggagaatcatatggatctgaaaagggtgctcctggagaccctggcctgcagggaaaacccggaaaagatggtgttcctggcttccctggaagtgagggagtcaagggcaacaggggtttccctgggttaatgggtgaagatggcattaagggacagaaaggggacattggccctccaggatttcgtggtccaacagaatattatgacacataccaggaaaagggagatgaaggcactccaggcccaccagggcccagaggagctcgtggcccacaaggtcccagtggtccccccggagttcctggaagtcctggatcatcaaggcctggcctcagaggagcccctggatggccaggcctgaaaggaagtaaaggggaacgaggccgcccaggaaaggatgccatggggactcctgggtccccaggttgtgctggttcaccaggtcttccaggatcaccgggacctccaggaccgccaggtgacatcgtttttcgcaagggtccacctggagatcacggactgccaggctatctagggtctccaggaatcccaggagttgatgggcccaaaggagaaccaggcctcctgtgtacacagtgcccttatatcccagggcctcccggtctcccaggattgccagggttacatggtgtaaaaggaatcccaggaagacaaggcgcagctggcttgaaaggaagcccagggtccccaggaaatacaggtcttccaggatttccaggtttcccaggtgcccagggtgacccaggacttaaaggagaaaaaggtgaaacacttcagcctgaggggcaagtgggtgtcccaggtgacccggggctcagaggccaacctgggagaaagggcttggatggaattcctggaactccgggagtgaaaggattaccaggacctaaaggcgaactggctctgagtggtgagaaaggggaccaaggtcctccaggggatcctggctcccctgggtccccaggacctgcaggaccagctggaccacctggctacggaccccaaggagaacctggtctccagggcacgcaaggagttcctggagcccccggaccacccggagaagccggccctaggggagagctcagtgtttcaacaccagttccaggcccaccaggacctccagggccccctggccatcctggcccccaaggtccacctggtatccctggatccctggggaaatgtggagatcctggtcttccagggcctgatggtgaaccaggaattccaggaattggatttcctgggcctcctggacctaagggagaccaaggttttccaggtacaaaaggatcactgggttgtcctggaaaaatgggagagcctgggttacctggaaagccaggcctcccaggagccaagggagaaccagcagtagccatgcctggaggaccaggaacaccaggttttccaggagaaagaggcaattctggggaacatggagaaattggactccctggacttccaggtctccctggaactccaggaaatgaagggcttgatggaccacgaggagatccagggcagcctggaccacctggagaacaaggacccccaggaaggtgcatagagggtcccaggggagcccaaggacttccaggcttaaatggattgaaagggcaacaaggcagaagaggtaaaacggggccaaagggagacccaggaattccaggcttggatagatcaggatttcctggagaaactggatcaccaggaattccaggtcatcaaggtgaaatgggaccactgggtcaaagaggatatccaggaaatccgggaattttagggccaccaggtgaagatggagtgattgggatgatgggctttcctggagccattggccctccagggccccctgggaacccaggcacaccagggcagagggggagccctggaattccaggagtaaagggccagagaggaaccccaggagccaagggggaacaaggagataaaggaaatcccgggccttcagagatatcccacgtaataggggacaaaggagaaccaggtctcaaaggattcgcaggaaatccaggtgagaaaggaaacagaggcgttccagggatgccaggtttaaagggcctcaaaggactacccggaccagcaggaccaccaggccccagaggagatttgggcagcactgggaatcctggagaaccaggactgcgtggtataccaggaagcatggggaacatgggcatgccaggttctaaaggaaaaaggggaactttgggattcccaggtcgagcaggaagaccaggcctcccaggtattcatggtctccagggagataagggagagccaggttattcagaaggtacaaggccaggaccaccgggaccaacgggggatccaggactgccgggtgatatgggaaagaaaggagaaatggggcaacctggcccacctggacatttggggcctgctggacctgagggagcccctggaagtcctggaagtcctggcctcccaggaaagccaggtcctcatggtgatttgggttttaaaggaatcaaaggcctcctgggccctccaggaatcagaggccctccaggtcttccaggatttccaggatctcctggaccaatgggtataagaggtgaccaaggacgtgatggaattcctggtccagccggagaaaagggagaaacgggtttattgagggcccctccaggcccaagagggaaccctggtgctcaaggagccaaaggagacaggggagccccaggttttcctggcctcccgggcagaaaaggggccatgggagatgctggacctcgaggacccacaggcatagaaggattcccagggccaccaggtctgcccggtgcaattatccctggccagacaggaaatcgtggtccaccaggctcaagaggaagcccaggtgcgcctggtccccctggacctccagggagtcatgtaataggcataaaaggagacaaagggtctatgggccaccctggcccaaaaggtccacctggaactgcaggagacatgggaccaccaggtcgtctgggagcaccaggtactccaggtcttccaggacccagaggtgatcctggattccaggggtttccaggcgtgaaaggagaaaagggtaatcctggatttctaggatccattggacctccaggaccaattgggccaaaaggaccacctggtgtacgtggagaccctggcacacttaagattatctcccttccaggaagcccagggccacctggcacacctggagaaccagggatgcagggagaacctgggccaccagggccacctggaaacctaggaccctgtgggccaagaggtaagccaggcaaggatggaaaaccaggaactcctggaccagctggagaaaaaggcaacaaaggttctaaaggagagccaggaccagctggatcagatggattgccaggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagacactca

SEQ ID NO: 2 - protein, 1671 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRHS

SEQ ID NO: 3 - DNA, 5073 bp, Homo sapiens
atgtggtctctgcacatagtactaatgaggtgctccttcagattgaccaagtccttggccacaggtccctggtcacttatactcattctcttttctgtacaatatgtatatgggagtggaaagaaatacattggtccttgtggaggaagagattgctctgtttgccactgtgttcctgaaaaggggtctcggggtccaccaggaccaccagggccacagggtccaattggacccctgggagccccaggacccattgggctttcaggagagaaaggaatgagaggggaccgcggccctcctggagcagcaggggacaaaggagataagggtccaactggtgttcctggatttccaggtttagatggcatacctgggcacccagggcctcctggacccagaggcaaacctggtatgagtggccacaatggctcaagaggtgacccagggtttccaggaggaagaggagctcttggcccaggaggccccctaggccatcctggggaaaagggagaaaaaggaaattcagtgttcattttaggtgccgttaaaggtattcagggagacagaggggacccaggactgcctggcttaccaggatcttggggtgcaggaggaccggcaggtcccacaggatatcctggagagccagggttagtgggacctccgggccaaccagggcgtccaggtttgaagggaaatcccggtgtgggagtaaaggggcaaatgggagacccgggtgaggttggtcagcaaggttctcctggacccaccctgttggtagagccacctgacttttgtctctataaaggagaaaagggtataaaaggaattcctggaatggttggactgccaggaccaccaggacgcaagggagaatctggtattggggcaaaaggagaaaaaggtattcctggatttccagggcctcggggggatcctggttcctatggatctccaggttttccaggattaaagggagaactaggactggttggagatcctgggctatttggattaattggcccaaagggggatcctggaaatcgagggcacccaggaccaccaggtgttttggtgactccacctcttccactcaaaggcccaccaggggacccagggttccctggccgctatggagaaacaggggatgttggaccacctggtcccccaggtctcttgggcagaccaggggaagcctgtgcaggcatgataggaccccctgggccacaaggatttcctggtcttcctgggcttccaggagaagctggtattcctgggagacctgattctgctccaggaaaaccagggaagccaggatcacctggcttgcctggagcaccaggcctgcagggcctcccaggatcaagtgtgatatactgtagtgttgggaaccccggaccacaaggaataaaaggcaaagttggtcccccaggaggaagaggcccaaaaggagaaaaaggaaatgaaggactctgtgcctgtgagcctggacccatgggcccccctggccctccaggacttcctgggaggcaggggagtaagggagacttggggctccctggctggcttggaacaaaaggtgacccaggacctcctggtgctgaaggacctccagggctaccaggaaagcatggtgcctctggaccacctggcaacaaaggggcgaagggtgacatggttgtatcaagagttaaagggcacaaaggagaaagaggtcctgatgggcccccaggatttccagggcagccaggatcacatggtcgggatggacatgctggagaaaaaggggatccaggacctccaggggatcatgaagatgcgaccccaggtggtaaaggatttcctggacctctgggccccccaggcaaagcaggacctgtggggcccccaggactgggatttcctggtccaccaggagagcgaggccacccaggagttccaggccacccaggtgtgaggggccctgatggcttgaagggtcagaaaggtgacacaatttcttgcaacgtaacctaccctgggaggcatggccctccaggttttgatggacctccaggtccgaagggatttccaggtccccaaggtgcccctgggctgagtggttcagatgggcataaaggcagacctggcacaccaggaacagcggaaataccaggtccacctggttttcgtggtgacatgggagatccgggttttggaggtgaaaaggggtcctcccctgttgggcccccaggccctcccggctcaccaggagtgaatggtcagaaaggaatcccgggagaccctgcatttggtcacctgggacccccgggaaagaggggtctttcaggagtgccagggataaaaggacccagaggtgatccgggatgtccaggggctgaagggccagctggcattcctggattcctaggtctcaaaggtcccaaaggcagagagggacatgctgggtttccaggtgtcccaggtccacctggccattcctgtgaaagaggtgctccagggataccagggcaaccgggactccctgggtatccaggtagcccaggtgctccaggtgggaaaggacagccgggagatgtggggcctcccgggccagctggaatgaaaggcctccccggactcccaggacggcctggggcacatggtcccccaggcctcccaggaatcccaggtccctttggagatgatgggctacctggtcctccaggtccaaagggaccccgggggctgcctggtttcccaggttttcccggagaaagaggaaagcctggtgcagagggatgtcctggcgcaaagggagaacctggagagaagggcatgtctggccttcctggagaccggggactgagaggggccaaaggagccataggacctcccggagatgaaggagaaatggctatcatttcacaaaagggaacacctggggaacctggacctcctggagatgatggattcccaggagaaagaggtgataaaggaactcccgggatgcaagggagaagaggagagccgggaagatacggaccacctggatttcacagaggggaacctggtgagaaaggtcagccagggcctcctggacccccaggccctccaggctcaactggtctaagagggttcattggttttccaggacttccaggtgaccagggtgagccaggttctccaggtccccctggattttcaggaattgatggagcaagaggacctaaaggaaacaaaggtgaccctgccagtcactttggtccacctggtccaaagggtgagccaggtagccctggatgtccagggcattttggagcatccggagagcagggcttgcctggtattcaagggcccagaggatcacctggaaggccagggccacctggctcctctggaccaccagggtgcccaggtgatcacgggatgcctgggctgaggggacagccaggagaaatgggagaccctgggccaagaggcctccagggggatccagggataccaggtcctccgggaataaaaggtccctccggatcacctggcctgaacggcttgcatggattgaaaggtcagaaaggaactaaaggtgcttcaggtttgcatgatgtggggccacctggtccagtgggaatacctgggctaaaaggggagagaggagaccctgggagcccaggaatctctcctccaggtcctcgtggaaagaaaggtcccccaggacccccagggagttcaggaccacctggtcctgcaggtgccacaggaagagctcctaaggacattcctgacccgggtccacctggagatcagggacctcctggtcctgatggcccaagaggagcacctgggcctccaggcctccctgggagtgttgaccttctgagaggggagccaggtgactgtggtctaccagggccaccaggtccccctggcccaccaggccctccaggatacaaaggctttccaggatgtgatggaaaagatggccagaaaggaccagtgggattcccgggaccgcagggaccacatggatttcctgggccacctggagagaagggtttacctggacctccagggagaaaagggcccactggtcttccgggtcccagaggtgaaccggggccacctgcagatgtggatgactgtccccgaatcccaggccttcctggggcgccaggcatgagaggaccagaaggagccatggggctccctggaatgagaggcccctcaggaccagggtgcaaaggagagcctgggctggatggcaggaggggtgtggatggcgtccctgggtctcctgggcctcccggacgtaaaggtgacacaggagaagacggctaccctggaggaccagggcctcctggtcccattggggatcctgggcccaaagggtttggccctggatacctcggtggcttcctcctggttctccacagtcagacggaccaggagcccacctgccccctgggcatgcccaggctctggactgggtatagtctgttatacctggaagggcaagagaaagctcacaatcaagaccttggtctggcagggtcttgccttcccgtatttagcacgctgccctttgcctactgcaacatccaccaggtgtgccactatgcccagagaaacgacagatcctactggctggccagcgctgcgcccctccccatgatgccactctctgaagaggcgatccgcccctatgtcagccgctgtgcggtatgcgaggccccggcccaggcggtggcggtgcacagccaggaccagtccatccccccatgtccgcagacctggaggagcctctggatcgggtattcattcctgatgcacacaggagctggggaccaaggaggagggcaggcccttatgtcacctggcagctgcctggaagatttcagagcagcaccattccttgaatgccagggccggcagggaacttgccactttttcgcaaataagtatagcttctggctcacaacggtgaaagcagacttgcagttttcctctgctccagcaccagacaccttaaaagaaagccaggcccaacgccagaaaatcagccggtgccaggtctgcgtgaagtatagcttg

SEQ ID NO: 4 - protein, 1691 aa, Homo sapiens
MWSLHIVLMRCSFRLTKSLATGPWSLILILFSVQYVYGSGKKYIGPCGGRDCSVCHCVPEKGSRGPPGPPGPQGPIGPLGAPGPIGLSGEKGMRGDRGPPGAAGDKGDKGPTGVPGFPGLDGIPGHPGPPGPRGKPGMSGHNGSRGDPGFPGGRGALGPGGPLGHPGEKGEKGNSVFILGAVKGIQGDRGDPGLPGLPGSWGAGGPAGPTGYPGEPGLVGPPGQPGRPGLKGNPGVGVKGQMGDPGEVGQQGSPGPTLLVEPPDFCLYKGEKGIKGIPGMVGLPGPPGRKGESGIGAKGEKGIPGFPGPRGDPGSYGSPGFPGLKGELGLVGDPGLFGLIGPKGDPGNRGHPGPPGVLVTPPLPLKGPPGDPGFPGRYGETGDVGPPGPPGLLGRPGEACAGMIGPPGPQGFPGLPGLPGEAGIPGRPDSAPGKPGKPGSPGLPGAPGLQGLPGSSVIYCSVGNPGPQGIKGKVGPPGGRGPKGEKGNEGLCACEPGPMGPPGPPGLPGRQGSKGDLGLPGWLGTKGDPGPPGAEGPPGLPGKHGASGPPGNKGAKGDMVVSRVKGHKGERGPDGPPGFPGQPGSHGRDGHAGEKGDPGPPGDHEDATPGGKGFPGPLGPPGKAGPVGPPGLGFPGPPGERGHPGVPGHPGVRGPDGLKGQKGDTISCNVTYPGRHGPPGFDGPPGPKGFPGPQGAPGLSGSDGHKGRPGTPGTAEIPGPPGFRGDMGDPGFGGEKGSSPVGPPGPPGSPGVNGQKGIPGDPAFGHLGPPGKRGLSGVPGIKGPRGDPGCPGAEGPAGIPGFLGLKGPKGREGHAGFPGVPGPPGHSCERGAPGIPGQPGLPGYPGSPGAPGGKGQPGDVGPPGPAGMKGLPGLPGRPGAHGPPGLPGIPGPFGDDGLPGPPGPKGPRGLPGFPGFPGERGKPGAEGCPGAKGEPGEKGMSGLPGDRGLRGAKGAIGPPGDEGEMAIISQKGTPGEPGPPGDDGFPGERGDKGTPGMQGRRGEPGRYGPPGFHRGEPGEKGQPGPPGPPGPPGSTGLRGFIGFPGLPGDQGEPGSPGPPGFSGIDGARGPKGNKGDPASHFGPPGPKGEPGSPGCPGHFGASGEQGLPGIQGPRGSPGRPGPPGSSGPPGCPGDHGMPGLRGQPGEMGDPGPRGLQGDPGIPGPPGIKGPSGSPGLNGLHGLKGQKGTKGASGLHDVGPPGPVGIPGLKGERGDPGSPGISPPGPRGKKGPPGPPGSSGPPGPAGATGRAPKDIPDPGPPGDQGPPGPDGPRGAPGPPGLPGSVDLLRGEPGDCGLPGPPGPPGPPGPPGYKGFPGCDGKDGQKGPVGFPGPQGPHGFPGPPGEKGLPGPPGRKGPTGLPGPRGEPGPPADVDDCPRIPGLPGAPGMRGPEGAMGLPGMRGPSGPGCKGEPGLDGRRGVDGVPGSPGPPGRKGDTGEDGYPGGPGPPGPIGDPGPKGFGPGYLGGFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYSL

SEQ ID NO: 5 - DNA, 5073 bp, Homo sapiens
atgaaactgcgtggagtcagcctggctgccggcttgttcttactggccctgagtctttgggggcagcctgcagaggctgcggcttgctatgggtgttctccaggatcaaagtgtgactgcagtggcataaaaggggaaaagggagagagagggtttccaggtttggaaggacacccaggattgcctggatttccaggtccagaagggcctccggggcctcggggacaaaagggtgatgatggaattccagggccaccaggaccaaaaggaatcagaggtcctcctggacttcctggatttccagggacaccaggtcttcctggaatgccaggccacgatggggccccaggacctcaaggtattcccggatgcaatggaaccaagggagaacgtggatttccaggcagtcccggttttcctggtttacagggtcctccaggaccccctgggatcccaggtatgaagggtgaaccaggtagtataattatgtcatcactgccaggaccaaagggtaatccaggatatccaggtcctcctggaatacaaggcctacctggtcccactggtataccagggccaattggtcccccaggaccaccaggtttgatgggccctcctggtccaccaggacttccaggacctaaggggaatatgggcttaaatttccagggacccaaaggtgaaaaaggtgagcaaggtcttcagggcccacctgggccacctgggcagatcagtgaacagaaaagaccaattgatgtagagtttcagaaaggagatcagggacttcctggtgaccgagggcctcctggacctccagggatacgtggtcctccaggtcccccaggtggtgagaaaggtgagaagggtgagcaaggagagccaggcaaaagaggtaaaccaggcaaagatggagaaaatggccaaccaggaattcctggtttgcctggtgatcctggttaccctggtgaacccggaagggatggtgaaaagggccaaaaaggtgacactggcccacctggacctcctggacttgtaattcctagacctgggactggtataactataggagaaaaaggaaacattgggttgcctgggttgcctggagaaaaaggagagcgaggatttcctggaatacagggtccacctggccttcctggacctccaggggctgcagttatgggtcctcctggccctcctggatttcctggagaaaggggtcagaaaggtgatgaaggaccacctggaatttccattcctggacctcctggacttgacggacagcctggggctcctgggcttccagggcctcctggccctgctggccctcacattcctcctagtgatgagatatgtgaaccaggccctccaggccccccaggatctccaggtgataaaggactccaaggagaacaaggagtgaaaggtgacaaaggtgacacttgcttcaactgcattggaactggtatttcagggcctccaggtcaacctggtttgccaggtctcccaggtcctccaggatctcttggtttccctggacagaaaggggaaaaaggacaagctggtgcaactggtcccaaaggattaccaggcattccaggagctccaggtgctccaggctttcctggatctaaaggtgaacctggtgatatcctcacttttccaggaatgaagggtgacaaaggagagttgggttcccctggagctccagggcttcctggtttacctggcactcctggacaggatggattgccagggcttcctggcccgaaaggagagcctggtggaattacttttaagggtgaaagaggtccccctgggaacccaggtttaccaggcctcccagggaatatagggcctatgggtccccctggtttcggccctccaggcccagtaggtgaaaaaggcatacaaggtgtggcaggaaatccaggccagccaggaataccaggtcctaaaggggatccaggtcagactataacccagccggggaagcctggcttgcctggtaacccaggcagagatggtgatgtaggtcttccaggtgaccctggacttccagggcaaccaggcttgccagggatacctggtagcaaaggagaaccaggtatccctggaattgggcttcctggaccacctggtcccaaaggctttcctggaattccaggacctccaggagcacctgggacacctggaagaattggtctagaaggccctcctgggccacccggctttccaggaccaaagggtgaaccaggatttgcattacctgggccacctgggccaccaggacttccaggtttcaaaggagcacttggtccaaaaggtgatcgtggtttcccaggacctccgggtcctccaggacgcactggcttagatgggctccctggaccaaaaggtgatgttggaccaaatggacaacctggaccaatgggacctcctgggctgccaggaataggtgttcagggaccaccaggaccaccagggattcctgggccaataggtcaacctggtttacatggaataccaggagagaagggggatccaggacctcctggacttgatgttccaggacccccaggtgaaagaggcagtccagggatccccggagcacctggtcctataggacctccaggatcaccagggcttccaggaaaagcaggtgcctctggatttccaggtaccaaaggtgaaatgggtatgatgggacctccaggcccaccaggacctttgggaattcctggcaggagtggtgtacctggtcttaaaggtgatgatggcttgcagggtcagccaggacttcctggccctacaggagaaaaaggtagtaaaggagagcctggccttccaggccctcctggaccaatggatccaaatcttctgggctcaaaaggagagaagggggaacctggcttaccaggtatacctggagtttcagggccaaaaggttatcagggtttgcctggagacccagggcaacctggactgagtggacaacctggattaccaggaccaccaggtcccaaaggtaaccctggtctccctggacagccaggtcttataggacctcctggacttaaaggaaccatcggtgatatgggttttccagggcctcagggtgtggaagggcctcctggaccttctggagttcctggacaacctggctccccaggattacctggacagaaaggcgacaaaggtgatcctggtatttcaagcattggtcttccaggtcttcctggtccaaagggtgagcctggtctgcctggatacccagggaaccctggtatcaaaggttctgtgggagatcctggtttgcccggattaccaggaacccctggagcaaaaggacaaccaggccttcctggattcccaggaaccccaggccctcctggaccaaaaggtattagtggccctcctgggaaccccggccttccaggagaacctggtcctgtaggtggtggaggtcatcctgggcaaccagggcctccaggcgaaaaaggcaaacccggtcaagatggtattcctggaccagctggacagaagggtgaaccaggtcaaccaggctttggaaacccaggaccccctggacttccaggactttctggccaaaagggtgatggaggattacctgggattccaggaaatcctggccttccaggtccaaagggcgaaccaggctttcacggtttccctggtgtgcagggtcccccaggccctcctggttctccgggtccagctctggaaggacctaaaggcaaccctgggccccaaggtcctcctgggagaccaggtcctacaggttttcaaggtctaccaggtccagaaggtcctccaggtctccctggaaatggaggtattaaaggagagaagggaaatccaggccaacctgggctacctggcttgcctggtttgaaaggagatcaaggaccaccaggactccagggtaatcctggccggccgggtctcaatggaatgaaaggagatcctggtctccctggtgttccaggattcccaggcatgaaaggacccagtggagtacctggatcagctggccctgagggggaaccgggacttattggtcctccaggtcctcctggattacctggtccttcaggacagagtatcataattaaaggagatgctggtcctccaggaatccctggccagcctgggctaaagggtctaccaggaccccaaggacctcaaggcttaccaggtccaactggccctccaggagatcctggacgcaatggactccctggctttgatggtgcaggagggcgcaaaggagacccaggtctgccaggacagccaggtacccgtggtttggatggtccccctggtccagatggattgcaaggtcccccaggtccccctggaacctcctctgttgcacatggatttcttattacacgccacagccagacaacggatgcaccacaatgcccacagggaacacttcaggtctatgaaggcttttctctcctgtatgtacaaggaaataaaagagcccacggtcaagacttggggacggctggcagctgccttcgtcgctttagtaccatgcctttcatgttctgcaacatcaataatgtttgcaactttgcttcaagaaatgactattcttactggctctctaccccagagcccatgccaatgagcatgcaacccctaaagggccagagcatccagccattcattagtcgatgtgcagtatgtgaagctccagctgtggtgatcgcagttcacagtcagacgatccagattccccattgtcctcagggatgggattctctgtggattggttattccttcatgatgcatacaagtgcaggggcagaaggctcaggtcaagccctagcctcccctggttcctgcttggaagagtttcgttcagctcccttcatcgaatgtcatgggaggggtacctgtaactactatgccaactcctacagcttttggctggcaactgtagatgtgtcagacatgttcagtaaacctcagtcagaaacgctgaaagcaggagacttgaggacacgaattagccgatgtcaagtgtgcatgaagaggaca

SEQ ID NO: 6 - protein, 1691 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGPTGFQGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 7 - DNA, 33 bp, Artificial Sequence
  Split Luciferase SmBiT
gtgaccggctaccggctgttcgaggagattctg

SEQ ID NO: 8 - protein, 11 aa, Artificial Sequence
  Synthetic Construct
VTGYRLFEEIL

SEQ ID NO: 9 - DNA, 477 bp, Artificial Sequence
  Split Luciferase LgBiT
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagt

SEQ ID NO: 10 - protein, 159 aa, Artificial Sequence
  Synthetic Construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 11 - DNA, 63 bp, Artificial Sequence
  Ig-kappa leader sequence
atggagacagacacactcctgctatgggtactgctgctctgggttccaggttccactggtgac

SEQ ID NO: 12 - protein, 8 aa, Artificial Sequence
  FLAG tag
DYKDDDDK

SEQ ID NO: 13 - protein, 22 aa, Artificial Sequence
  3xFLAG tag
DYKDHDGDYKDHDIDYKDDDDK

(19) United States
(12) Patent Application Publication
(10) Pub. No.: US 2020/0172956 A1
(43) Pub. Date: Jun. 4, 2020

(54) Evaluation System for Therapeutic Drug for Genetic Kidney Disorder Alport Syndrome

(71) Applicant: NATIONAL UNIVERSITY CORPORATION KUMAMOTO UNIVERSITY, KUMAMOTO-SHI, KUMAMOTO (JP)

(72) Inventors: HIROFUMI KAI, KUMAMOTO (JP); TSUYOSHI SHUTO, KUMAMOTO (JP); MARY ANN SUICO, KUMAMOTO (JP); KOHEI OMACHI, KUMAMOTO (JP)

(21) Appl. No.: 16/614,531

(22) PCT Filed: May 18, 2018
(86) PCT No.: PCT/JP2018/019283

§ 371 Date: Nov. 18, 2019

(30) Foreign Application Priority Data
May 19, 2017 (JP) 2017-099497

(51) Int. Cl. C12Q 1/66; C07K 14/78; C12N 9/02; C12N 15/63; G01N 21/76

(52) U.S. Cl. CPC C12Q 1/66; C07K 14/78; C12N 9/0069; G01N 2333/90241; G01N 21/763; C07K 2319/43; C12N 15/63

(57) ABSTRACT

The present invention relates to a method for evaluating a potential of type IV collagen trimerization, a method of screening for a compound that promotes a potential of type IV collagen trimerization, and kits for use with these methods. Because the potential of type IV collagen trimerization is associated with the onset of Alport syndrome, the methods and the kits of the present invention can be powerful tools in drug development and/or diagnosis.

### TECHNICAL FIELD

The present invention relates to a method for evaluating a potential of type IV collagen trimerization, a method of screening for a compound that promotes a potential of type IV collagen trimerization, a method for evaluating an effect of a compound that promotes a potential of type IV collagen trimerization, and kits for use with these methods.

### BACKGROUND ART

Alport syndrome is a hereditary disease caused by mutation in type IV collagen (a3, a4, and a5(IV)), leading to a glomerular basement membrane anomaly and thus the onset of progressive nephritis. A past clinical study reports that patients, whose type IV collagen expression is found even a little on a basement membrane, have a mild symptom (NPL 1). A basic research report shows that pathology can be improved by postnatal re-expression of α3(IV) in a genetically deficient model mouse (NPL 2). Therapy using, as a target, causative α3, α4, and/or α5(IV) protein by itself should be feasible.

Meanwhile, the present inventors have revealed that the wild-type and a mutant α5(IV) do not have a difference in intracellular stability and even the mutant is relatively stable inside cells. The results suggest that for therapy using α3, α4, and/or α5(IV) as a target, it is important to promote and restore lost trimerization but not to promote stability by, for instance, inhibition of protein degradation.

To date, no method has been known that quantitatively evaluates trimerization of α3, α4, and α5 chains of type IV collagen. Here, detection using immunoprecipitation for detecting a complex has already been tried. However, the reproducibility and quantitativity are low. Hence, it is difficult to use the detection in screening for a compound that promotes trimerization.

### CITATION LIST

### Non Patent Literature

NPL 1: Hashimura, Y., et al., Kidney Int., 2014, 85(5): 1208-1213
NPL 2: Lin, X., et al., J. Am. Soc. Nephroi., 2014, 25(4): 687-692

### SUMMARY OF INVENTION

### Technical Problem

The present invention provides a method for evaluating a potential of type IV collagen trimerization, a method of screening for a compound that promotes a potential of type IV collagen trimerization, a method for evaluating an effect of a compound that promotes a potential of type IV collagen trimerization, and kits for use with these methods.

### Solution to Problem

In view of the above, the present inventors have started research while focusing on trimerization of type IV collagen, and after intensive investigation, have established an in vitro assay system, based on a luciferase, that evaluates trimerization of type IV collagen. Based on the findings, the present invention has been completed.

Specifically, an aspect of the present invention is as follows.

[1] A method for evaluating a potential of type IV collagen trimerization, comprising

(1) culturing cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof, and

(3) evaluating a potential of type IV collagen trimerization in accordance with a luminescence emission intensity.

[2] The method according to [1], wherein the cells co-expressing the fusion proteins (a) to (c) are obtained by transfecting a cell with:

(a′) an expression vector comprising a gene encoding a fusion protein comprising a wild-type or mutant type IV collagen α3(IV) chain and one of split luciferase fragments;

(b′) an expression vector comprising a gene encoding a fusion protein comprising a wild-type or mutant type IV collagen α4(IV) chain and a peptide tag; and

(c′) an expression vector comprising a gene encoding a fusion protein comprising a wild-type or mutant type IV collagen α5(IV) chain and the other split luciferase fragment.

[3] The method according to [1] or [2], wherein the fusion proteins (a) to (c) are:

(a) a fusion protein comprising one of split luciferase fragments on a C-terminal side of a wild-type or mutant type IV collagen α3(IV) chain and;

(b) a fusion protein comprising a peptide tag on a C-terminal side of a wild-type or mutant type IV collagen α4(IV) chain; and

(c) a fusion protein comprising the other split luciferase fragment on a C-terminal side of a wild-type or mutant type IV collagen α5(IV) chain.

[4] The method according to [1] or [2], wherein the fusion proteins (a) to (c) are:

(a) a fusion protein comprising one of split luciferase fragments on an N-terminal side of a wild-type or mutant type IV collagen α3(IV) chain and;

(b) a fusion protein comprising a peptide tag on a C-terminal side of a wild-type or mutant type IV collagen α4(IV) chain; and

(c) a fusion protein comprising the other split luciferase fragment on an N-terminal side of a wild-type or mutant type IV collagen α5(IV) chain.

[5] The method according to any one of [1] to [4], wherein the peptide tag is FLAG tag (SEQ ID NO: 12) or 3×FLAG tag (SEQ ID NO: 13).

[6] A method of screening for a compound that promotes a potential of type IV collagen trimerization, comprising

(1) culturing, in the presence or absence of a candidate compound, cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof,

(3) comparing a luminescence emission intensity of the culture product cultured in the presence of the candidate compound with a luminescence emission intensity of the culture product cultured in the absence of the candidate compound, and

(4) identifying the candidate compound as a compound that promotes a potential of type IV collage trimerization when the luminescence emission intensity of the culture product cultured in the presence of the candidate compound is higher than the luminescence emission intensity of the culture product cultured in the absence of the candidate compound.

[7] A method for evaluating an effect of a compound that promotes a potential of type IV collagen trimerization, comprising

(1) culturing, in the presence of each serially diluted candidate compound, cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof, and

(3) evaluating, based on a luminescence emission intensity according to a concentration of the candidate compound, concentration dependency of the candidate compound with regard to promoting a potential of type IV collagen trimerization.

[8] A method for evaluating an effect of a compound that promotes a potential of type IV collagen trimerization, comprising

(1) culturing, in the presence of each of a plurality of candidate compounds, cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof, and

(3) measuring a luminescence emission intensity in the presence of each candidate compound to determine a candidate compound exhibiting a higher luminescence emission intensity as a compound with a higher effect of promoting a potential of type IV collagen trimerization.

[9] A kit for evaluating a potential of type IV collagen trimerization, screening for a compound that promotes a potential of type IV collagen trimerization, or evaluating a therapeutic drug for Alport syndrome, the kit comprising:

(a′) an expression vector comprising a gene encoding a fusion protein comprising a wild-type or mutant type IV collagen α3(IV) chain and one of split luciferase fragments;

(b′) an expression vector comprising a gene encoding a fusion protein comprising a wild-type or mutant type IV collagen α4(IV) chain and a peptide tag; and

(c′) an expression vector comprising a gene encoding a fusion protein comprising a wild-type or mutant type IV collagen α5(IV) chain and the other split luciferase fragment.

[10] A kit for evaluating a potential of type IV collagen trimerization, screening for a compound that promotes a potential of type IV collagen trimerization, or evaluating a therapeutic drug for Alport syndrome, the kit comprising cells co-expressing:

(a) a fusion protein comprising a wild-type or mutant type IV collagen α3(IV) chain and one of split luciferase fragments;

(b) a fusion protein comprising a wild-type or mutant type IV collagen α4(IV) chain and a peptide tag; and

(c) a fusion protein comprising a wild-type or mutant type IV collagen α5(IV) chain and the other split luciferase fragment.

### Advantageous Effects of Invention

A method for evaluating a potential of type IV collagen trimerization according to the present invention is a quantitative and highly reproducible method. Further, the method is applicable to high-throughput screening and can thus be utilized in screening for a compound that promotes a potential of type IV collagen trimerization. In addition, a method of the present invention can be used for evaluating an effect of a compound that promotes a potential of type IV collagen trimerization.

### BRIEF DESCRIPTION OF DRAWINGS

FIG. 1 is a schematic diagram of type IV collagen trimer detection by protein-protein interaction analysis using split luciferase fragments.

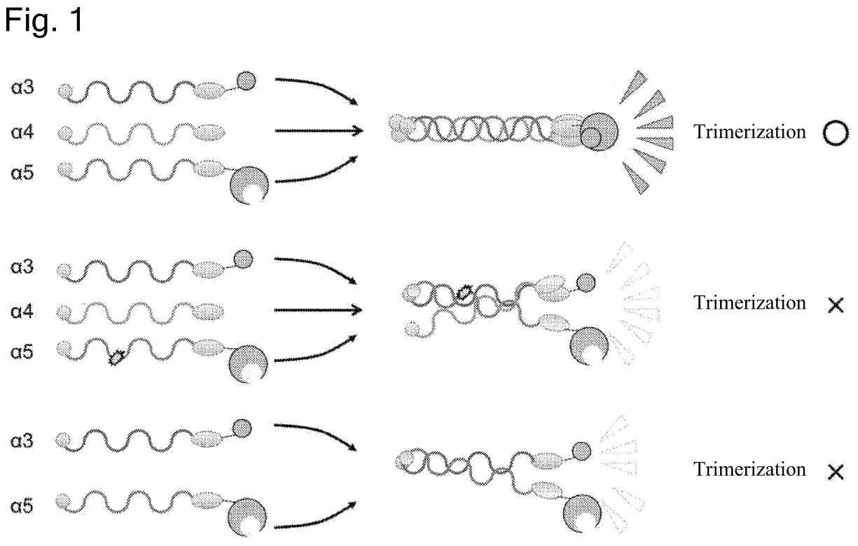

FIG. 2 is a schematic diagram of fusion proteins used in an assay system of Example 1 and a graph showing the results of detecting trimerization of the wild-type type IV collagen in this assay system. In the graph, α3 represents the results with a culture supernatant of α3 chain single-expression cells; α5 represents the results with a culture supernatant of α5 chain single-expression cells; α35 represents the results with a culture supernatant of cells co-expressing α3 and α5 chains; and α345 represents the results with a culture supernatant of cells co-expressing α3, α4, and α5 chains.

FIG. 3 is a graph showing the results of evaluating the amount of type IV collagen trimerization when the level of expression of wild-type type IV collagen α4 chain was changed. In the graph, α35 represents the results with a culture supernatant of cells co-expressing α3 and α5 chains; and α345 represents the results with a culture supernatant of cells co-expressing α3, α4, and α5 chains. The triangular bar shown over α4 schematically indicates the level of expression of α4 chain.

FIG. 4 is a schematic diagram of domain-deleted α5 chain fusion proteins and a graph showing the results of evaluating the amount of type IV collagen trimerization when the domain-deleted α5 chains were used.

FIG. 5 is a graph of evaluating type IV collagen trimerization in a culture supernatant when cells singly expressing each of α3 chain, α4 chain, and α5 chain or these three types of cells were co-cultured. In the graph, α3 represents the results with a culture supernatant of α3 chain single-expression cells; α4 represents the results with a culture supernatant of α4 chain single-expression cells; α5 represents the results with a culture supernatant of α5 chain single-expression cells; α3α4α5 represents the results with a culture supernatant from a co-culture of α3 chain single-expression cells, α4 chain single-expression cells, and α5 chain single-expression cells; and α345 represents the results with a culture supernatant of cells co-expressing α3, α4, and α5 chains.

FIG. 6 is a graph showing a trimerization pattern when various α5 chain mutants were used.

FIG. 7 is a schematic diagram of an evaluation system when using fusion proteins, in which a split luciferase fragment is fused on the N-terminal side of α3 or α5 chain, and a graph showing the results. In the graph, α3 represents the results with a culture supernatant of α3 chain single-expression cells; α5 represents the results with a culture supernatant of α5 chain single-expression cells; α35 represents the results with a culture supernatant of cells co-expressing α3 and α5 chains; and α345 represents the results with a culture supernatant of cells co-expressing α3, α4, and α5 chains.

### DESCRIPTION OF EMBODIMENTS

Hereinafter, the present invention is specifically described, but the present invention is not limited to them. Unless otherwise defined herein, scientific and technical terms pertained to and used for the present invention have meanings generally understood by those skilled in the art.

Method for Evaluating Potential of Type IV Collagen Trimerization

The present invention relates to a method for evaluating a potential of type IV collagen trimerization, comprising

(1) culturing cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof, and

(3) evaluating a potential of type IV collagen trimerization in accordance with a luminescence emission intensity.

Wild-type type-IV collagen α3(IV) chain (hereinafter, sometimes herein referred to as α3 chain) is a protein consisting of the amino acid sequence set forth in SEQ ID NO: 2 and is encoded by the nucleotide sequence set forth in SEQ ID NO: 1. Wild-type type-IV collagen α4(IV) chain (hereinafter, sometimes herein referred to as α4 chain) is a protein consisting of the amino acid sequence set forth in SEQ ID NO: 4 and is encoded by the nucleotide sequence set forth in SEQ ID NO: 3. Wild-type type-IV collagen α5(IV) chain (hereinafter, sometimes herein referred to as α5 chain) is a protein consisting of the amino acid sequence set forth in SEQ ID NO: 6 and is encoded by the nucleotide sequence set forth in SEQ ID NO: 5.

Mutant α3, α4, and α5 chains are α3, α4, and α5 chains having one or more point mutations in the amino acid sequences of wild-type α3, α4, and α5 chains, respectively. Each point mutation in the wild-type amino acid sequences may be selected from mutations found in patients with Alport syndrome, mutations found in patients suspected of Alport syndrome, or mutations identified of or suspected of relating to Alport syndrome after the filing of the present application. For instance, X-linked Alport syndrome, a causative gene of which is an α5 chain-encoding gene (COL4A5), accounts for about 80% of Alport syndrome. Examples of known α5 chain mutations related to Alport syndrome include G129E, G153D, G227S, G325R, G426R, G475S, G521D, G573D, G594D, G594S, G624D, G650D, L664N, G675S, G796D, G796R, G869R, G911E, S916G, G953V, G1030S, G1107R, G1143D, G1170S, G1220D, G1241C, G1241V, G1244D, G1448R, P1517T, C1567R, R1569Q, M1607I, L1649R, and R1683Q. Mutation G869R of α5 chain is the most frequently found mutation in patients with Alport syndrome and is preferable. Here, each point mutation is denoted by “X₁nX₂”; and n indicates the position of an amino acid in a wild-type sequence and, for α5 chain, agrees with the amino acid number of SEQ ID NO: 6. X₁ indicates an amino acid in a wild-type sequence; and X₂ indicates an amino acid in a mutated sequence. X₁ and X₂ are each expressed by amino acid one letter code well-known to those skilled in the art.

As used herein, the wording “having one or more point mutations” means that respective α3, α4, or α5 chain has 1 to 20, 1 to 15, 1 to 10, 1 to 5, or 1 to 3 point mutations.

The split luciferase refers to a pair of luciferase protein fragments encoded by two luciferase DNA sequences that have been split at a suitable site. A phenomenon is known that when these two split protein fragments come closer, activity of luciferase is restored and luminescence emission from a luminescent substrate can be retrieved. This phenomenon can be utilized to carry out a binding assay using, as an indicator, luminescence emission from a luciferase while split luciferase fragments are fused to respective molecules, association and/or polymer formation of which are to be observed, so as to form a pair. Examples of the split luciferase fragments that can be preferably used in the methods of the present invention include, but are not particularly limited to, a combination of SmBiT having the amino acid sequence of SEQ ID NO: 8 and encoded by the nucleotide sequence of SEQ ID NO: 7 and LgBiT having the amino acid sequence of SEQ ID NO: 10 and encoded by the nucleotide sequence of SEQ ID NO: 9. Which of SmBiT and LgBiT is fused to α3 chain or α5 chain is not particularly limited. Preferably, SmBiT is fused to α3 chain and LgBiT is fused to α5 chain.

Each split luciferase pair fragment may be fused on the C-terminal side or the N-terminal side of α3 chain or α5 chain. When each split luciferase pair fragment is fused on the N-terminal side of α3 chain or α5 chain, fusion proteins may be prepared such that the split luciferase pair fragment is inserted in a region after a signal sequence of α3 chain or α5 chain. In this case, the signal sequence used may be the signal sequence of α3 chain or α5 chain or may be replaced by another sequence known as a secretory protein-derived signal sequence. Examples of the other sequence available as a signal sequence include Igκ leader sequence (the sequence encoded by SEQ ID NO: 11) and IL-6 signal sequence.

The α4 chain is prepared as a fusion protein with a peptide tag. The peptide tag is not particularly limited in the art as long as the tag is used for the purpose of making easy recovery and/or detection of other proteins. When the molecular weight of the tag is large, it seems to prevent the split luciferase fragments from coming close to each other. From this viewpoint, the molecular weight of the peptide tag may be 15 kDa or less, 10 kDa or less, 5 kDa or less, or 3 kDa or less. For instance, FLAG tag (SEQ ID NO: 12) or 3×FLAG tag (SEQ ID NO: 13) may be suitably used as the peptide tag. In addition, it is preferable that the peptide tag is fused on the C-terminal side of α4 chain.

In a method of the present invention, cells co-expressing the fusion proteins (a), (b), and (c) may be obtained by transfecting a cell with: (a′) an expression vector comprising a gene encoding a fusion protein comprising a wild-type or mutant α3 chain and one of split luciferase fragments; (b′) an expression vector comprising a gene encoding a fusion protein comprising a wild-type or mutant α4 chain and a peptide tag; and (c′) an expression vector comprising a gene encoding a fusion protein comprising a wild-type or mutant α5 chain and the other split luciferase fragment.

The expression vectors (a′), (b′), and (c′) are not particularly limited if the vectors allow for expression of the fusion proteins (a), (b), and (c), respectively, when introduced into cells. The kind of the expression vectors may be selected, by those skilled in the art, depending on the kind of fusion protein-expressing cells containing the fusion proteins (a), (b), and (c). A technique known to those skilled in the art may be used to subclone, into a vector, a gene encoding each of the fusion proteins (a), (b), and (c).

The kind of cells is not particularly limited. Preferred are human-derived cells. Particularly preferred may be human kidney cells. For instance, HEK293T cells may be suitably used.

The transfection procedure is not particularly limited if the fusion proteins (a), (b), and (c) can be transiently expressed by introducing the expression vectors, and may be performed by a technique known to those skilled in the art.

Step (1) of the above method is a step of culturing cells co-expressing fusion proteins (a), (b), and (c). The culture medium and the culture condition may be suitably selected, by those skilled in the art, depending on the kind of cells. Examples of the available culture medium include DMEM, MEM, and RPMI-1640. When human-derived cells are used, it is preferable to use a serum-free culture medium. The culture condition is not particularly limited if the condition allows for growth of cells and may be, for instance, 5% CO₂ and 37° C. The culturing period may be from 24 to 72 h. It is preferable to use a phenol red-free culture medium during the last 24 h culturing.

The culturing of step (1) causes the fusion proteins (a), (b), and (c) to be expressed inside each cell. Next, these fusion proteins form a trimer to produce type IV collagen, which is then secreted outside each cell.

Step (2) of the above method is a step of adding a luminescent substrate to a culture product of step (1) and carrying out an incubation thereof. Because the intracellularly formed trimer type IV collagen is secreted outside each cell, the culture product of step (1) is preferably a culture supernatant. The luminescent substrate is not particularly limited if luminescence is emitted by a luciferase reaction. The incubation is not particularly limited if the luciferase reaction proceeds at the temperature and may be performed at from 30° C. to 40° C. and preferably 37° C. The incubation period is not particularly limited as long as the incubation is conducted within a range in which the amount of the trimer and the luminescence emission intensity caused by the luciferase reaction are proportionally correlated, and may be, for instance, within 10 min, within 15 min, or within 20 min.

Step (3) of the above method is a step of evaluating a potential of type IV collage trimerization in accordance with a luminescence emission intensity caused by the incubation of step (2). The luminescence emission intensity may be measured by a technique known, as a luciferase activity measurement, to those skilled in the art. It can be determined that as the luminescence emission intensity becomes stronger, the potential of trimerization of α3, α4, α5 chains used increases. For instance, a mutant may be used for either α3, α4, or α5 chain. In this case, by comparing the luminescence emission intensity when a wild-type α3, α4, or α5 chain is used, it is possible to evaluate a potential of trimerization compared with that of the wild-type.

Method of Screening for Compound that Promotes Potential of Type IV Collagen Trimerization,

The present invention relates to a method of screening for a compound that promotes a potential of type IV collagen trimerization, comprising

(1) culturing, in the presence or absence of a candidate compound, cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof,

(3) comparing a luminescence emission intensity of the culture product cultured in the presence of the candidate compound with a luminescence emission intensity of the culture product cultured in the absence of the candidate compound, and

(4) identifying the candidate compound as a compound that promotes a potential of type IV collage trimerization when the luminescence emission intensity of the culture product cultured in the presence of the candidate compound is higher than the luminescence emission intensity of the culture product cultured in the absence of the candidate compound.

In the above screening method, at least one of the fusion proteins (a), (b), and (c) contains a mutant α3, α4, or α5 chain.

The description about the following configuration is as described above in the section “Method for Evaluating Potential of Type IV Collagen Trimerization”.
Wild-type or mutant α3, α4, and α5 chains
Split luciferase fragments
Peptide tag
Cells co-expressing fusion proteins (a), (b), and (c); expression vectors used when the cells are prepared; and a procedure for preparing the cells (a transfection procedure).

Step (1) of the above screening method is a step of culturing, in the presence or absence of a candidate compound, cells co-expressing fusion proteins (a), (b), and (c). Except for addition of the candidate compound, the culture medium and the culture condition are as described above in the section “Method for Evaluating Potential of Type IV Collagen Trimerization”.

The candidate compound is a compound to be examined with respect to whether or not the candidate compound promotes a potential of type IV collagen trimerization. The concentration of the candidate compound is not particularly limited and the candidate compound may be present in the culture medium in a range from 1 μM to 100 mM, 5 μM to 50 mM, 7 μM to 30 mM, or 10 μM to 15 mM.

Step (2) of the above screening method is as described above with respect to step (2) of “Method for Evaluating Potential of Type IV Collagen Trimerization”.

Steps (3) and (4) of the above screening method are: steps of (3) comparing a luminescence emission intensity of the culture product cultured in the presence of the candidate compound with a luminescence emission intensity of the culture product cultured in the absence of the candidate compound, and (4) identifying the candidate compound as a compound that promotes a potential of type IV collage trimerization when the luminescence emission intensity of the culture product cultured in the presence of the candidate compound is higher than the luminescence emission intensity of the culture product cultured in the absence of the candidate compound. In a method of the present invention, it can be determined that as the luminescence emission intensity becomes higher, the effect of promoting a potential of trimerization of α3, α4, α5 chains used increases. Thus, when the luminescence emission intensity in the presence of the candidate compound is raised, it is possible to identify the candidate compound as a compound that exerts an effect of increasing a potential of trimerization of α3, α4, α5 chains used, namely a compound that promotes a potential of type IV collagen trimerization.

In addition, in the screening method of the present invention, by comparison with the luminescence emission intensity when fusion proteins including wild-type α3, α4, α5 chains are expressed in the absence of the candidate compound, it is possible to evaluate to what extent, as a result of the candidate compound promoting the potential of type IV collagen trimerization, this potential can be made close to the potential of wild-type type-IV collagen trimerization.

Method for Evaluating Effect of Compound that Promotes Potential of Type IV Collagen Trimerization

The present invention relates to a method for evaluating an effect of a compound that promotes a potential of type IV collagen trimerization (hereinafter, sometimes referred to as an evaluation method of the present invention).

A first embodiment of the evaluation method of the present invention may be a method comprising

(1) culturing, in the presence of each serially diluted candidate compound, cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof, and

(3) evaluating, based on a luminescence emission intensity according to a concentration of the candidate compound, concentration dependency of the candidate compound with regard to promoting a potential of type IV collagen trimerization.

A second embodiment of the evaluation method of the present invention may be a method comprising

(1) culturing, in the presence of each of a plurality of candidate compounds, cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,

(2) adding a luminescent substrate to a culture product of (1) and carrying out an incubation thereof, and

(3) measuring a luminescence emission intensity in the presence of each candidate compound to determine a candidate compound exhibiting a higher luminescence emission intensity as a compound with a higher effect of promoting a potential of type IV collagen trimerization.

In the above evaluation method procedure of the present invention, at least one of the fusion proteins (a), (b), and (c) contains a mutant α3, α4, or α5 chain.

The description about the following configuration for the evaluation method of the present invention is as described above in the section “Method for Evaluating Potential of Type IV Collagen Trimerization”.
Wild-type or mutant α3, α4, and α5 chains
Split luciferase fragments
Peptide tag
Cells co-expressing fusion proteins (a), (b), and (c); expression vectors used when the cells are prepared; and a procedure for preparing the cells (a transfection procedure).

In the evaluation method of the present invention, each candidate compound is a compound to be evaluated with respect to an effect of promoting a potential of type IV collagen trimerization. For instance, the compound may be identified by the screening method of the present invention or the compound may be used for treatment of Alport syndrome. The concentration of each candidate compound is not particularly limited and the candidate compound may be present in the concentration range described above in the section “Method of Screening for Compound That Promotes Potential of Type IV Collagen Trimerization”.

Step (2) in the evaluation method of the present invention is as described above in step (2) of “Method for Evaluating Potential of Type IV Collagen Trimerization”.

In the first embodiment of the above evaluation method, the concentration of a therapeutic drug present in step (1) is varied. In this way, it is possible to evaluate concentration dependency with regard to an effect of promoting a potential of type IV collagen trimerization by the candidate compound. Specifically, step (3) is a step of evaluating, based on a luminescence emission intensity according to a concentration of the candidate compound, concentration dependency of the candidate compound with regard to promoting a potential of type IV collagen trimerization. In a method of the present invention, it can be determined that as the luminescence emission intensity becomes higher, the effect of promoting a potential of trimerization of α3, α4, α5 chains used increases. Evaluation of the correlation between the concentration and the luminescence emission intensity of a candidate compound makes it possible to determine and identify a concentration range of the candidate compound required for promoting a potential of type IV collagen trimerization.

In the second embodiment of the above evaluation method, a plurality of candidate compounds are evaluated in parallel. In this way, it is possible to compare an effect of promoting a potential of type IV collagen trimerization among the candidate compounds. Specifically, step (3) is a step of measuring a luminescence emission intensity in the presence of each candidate compound to determine a candidate compound exhibiting a higher luminescence emission intensity as a compound with a higher effect of promoting a potential of type IV collagen trimerization. The luminescence emission intensities of the respective candidate compounds are compared. In this way, it is possible to relatively determine an effect of promoting a potential of type IV collagen trimerization between the candidate compounds.

In the second embodiment of the above evaluation method, each of the plurality of candidate compounds may be further serially diluted to prepare samples for usage. In this case, for each of the plurality of candidate compounds, it is possible to compare the effect of promoting a potential of type IV collagen trimerization and the concentration dependency among the candidate compounds.

For the evaluation method of the present invention, it may be possible to further compare the luminescence emission intensities when fusion proteins including wild-type α3, α4, α5 chains are expressed in the absence of each candidate compound. This comparison makes it possible to evaluate to what extent the potential of each mutant type IV collagen examined can be made close to the potential of wild-type type-IV collagen trimerization.

Any compound verified, by the screening method and the evaluation method of the present invention, to promote a potential of type IV collagen trimerization may be a compound useful as a therapeutic drug for Alport syndrome.

Kit

The present invention relates to a kit for evaluating a potential of type IV collagen trimerization, screening for a compound that promotes a potential of type IV collagen trimerization, or evaluating an effect of a compound that promotes a potential of type IV collagen trimerization, the kit comprising:

(a′) an expression vector comprising a gene encoding a fusion protein comprising a wild-type or mutant type IV collagen (IV) chain and one of split luciferase fragments;

(b′) an expression vector comprising a gene encoding a fusion protein comprising a wild-type or mutant type IV collagen α4(IV) chain and a peptide tag; and

(c′) an expression vector comprising a gene encoding a fusion protein comprising a wild-type or mutant type IV collagen α5(IV) chain and the other split luciferase fragment.

The present invention also relates to a kit for evaluating a potential of type IV collagen trimerization, screening for a compound that promotes a potential of type IV collagen trimerization, or evaluating an effect of a compound that promotes a potential of type IV collagen trimerization, the kit comprising cells co-expressing:

(a) a fusion protein comprising a wild-type or mutant type IV collagen α3(IV) chain and one of split luciferase fragments;

(b) a fusion protein comprising a wild-type or mutant type IV collagen α4(IV) chain and a peptide tag; and

(c) a fusion protein comprising a wild-type or mutant type IV collagen α5(IV) chain and the other split luciferase fragment.

The expression vectors (a′), (b′), and (c′) and the fusion proteins (a), (b), and (c) are as described above in the section “Method for Evaluating Potential of Type IV Collagen Trimerization”.

The kit of the present invention may comprise all the above components in one kit. The kit may aim at use in the methods of the present invention (a method for evaluating a potential of type IV collagen trimerization, a method of screening for a compound that promotes a potential of type IV collagen trimerization, or a method for evaluating an effect of a compound that promotes a potential of type IV collagen trimerization). This kit does not necessarily comprise part of the above components. If the kit does not comprise part of the above components, a practitioner can add any necessary component(s) to the kit so as to put into practice the method(s) of the present invention.

The kit of the present invention may comprise any additional component(s) including a culture medium and/or a luminescent substrate. The additional component(s) may be included as one kit in the kit of the present invention or may be provided as another kit, which is assumed to be used with the kit of the present invention.

The kit of the present invention may comprises a package insert in which instructions for performing the method(s) of the present invention are described, The package insert may describe, as descriptions, the matters set forth in the above sections “Method for Evaluating Potential of Type IV Collagen Trimerization”, “Method of Screening for Compound That Promotes Potential of Type IV Collagen Trimerization”, and “Method for Evaluating Effect of Compound That Promotes Potential of Type IV Collagen Trimerization”.

### EXAMPLES

Hereinafter, the present invention is specifically described by using Examples, but the present invention is not limited to them.

### Example 1: Type IV Collagen Formation Assay

Experimental Materials and Experimental Procedure

(1) Cell Type

In this study, HEK293T (Human Embryonic Kidney 293) cells were used. The HEK293T cells were purchased from the RIKEN CELL BANK.

(2) Cell Culturing Protocol

All tools used for culturing were autoclaved or subjected to sterilization treatment by dry-heat sterilization. For preparation of solutions, aqueous injection (OTSUKA DISTILLED WATER) or pure water prepared with an Elix pure water system (MILLIPORE) was used. In addition, all manipulations were conducted aseptically in a clean bench.

(3) Culture Medium

Basic Culture Medium: DMEM (Wako) was used, as a basic culture medium, for culturing HEK293T cells.

Culture Medium for Cell Growth: A basic culture medium supplemented with 10% fetal calf serum and antibiotics (penicillin G (100 units/mL) and streptomycin (100 μg/mL)) was used as a culture medium for cell growth.

(4) Culturing

The cells were subjected to stationary culture at 5% CO₂ and 37° C. in the culture medium for cell growth.

(5) Construction of DNA Plasmids (for α3 Chain and α5 Chain, C-terminal Side of Which Was Fused to Split Luciferase Fragment)

Each of a nucleic acid encoding wild-type type-IV collagen α3(IV) chain (COL4A3/SEQ ID NO: 1) and a nucleic acid encoding wild-type type-IV collagen α5(IV) chain (COL4A5/SEQ ID NO: 5) was subcloned into a nanoBiT plasmid (Promega). A nucleic acid encoding wild-type type-IV collagen α4(IV) chain (COL4A4/SEQ ID NO: 3) was subcloned into a pEB Multi Hyg (Wako). Here, the respective expression vectors after the subcloning were subcloned and contained: a nucleic acid encoding a fusion protein having one of split luciferase fragments at the C-terminal of type IV collagen α3(IV) chain (herein, also referred to as α3 chain); a nucleic acid encoding a fusion protein having the other split luciferase fragment at the C-terminal of type IV collagen α5(IV) chain (herein, also referred to as α5 chain); and a nucleic acid encoding a fusion protein having a FLAG tag at the C-terminal of type IV collagen α4(IV) chain (herein, also referred to as α4 chain).

The following Table 1 shows combinations of each gene and a plasmid containing the gene.

TABLE 1

| | | From which | Fusion protein |
|---|---|---|---|
| | | vendor the | that the expression |
| Gene | | vector was | vector after the |
| name | Vector | obtained | subcloning encodes |
| COL4A3 | pFC36K SmBiT | Promega | α3-SmBiT |
| COL4A5 | pFC34K LgBiT | Promega | α5-LgBiT |
| COL4A4 | pEB Multi Hyg | Wako | α4-FLAG |

(6) Transfection Protocol

For transfection with each gene in this experiment, TransiT-LT1 (Minis) was used to perform lipofection. The following shows the protocol.

First, an appropriate amount of TransIT-LT1 was added to 100 μL of a serum-free culture medium (Opti-MEM). The TransIT-LT1 was used such that the ratio of total DNA:TransIT-LT1 solution was 1:3 μL. Next, a gene of interest (0.5 to 2.0 μg) was added and mixed, and after mixing, the mixture was reacted for 15 min. Then, the mixed solution was added dropwise to subconfluent cultured cells, and the cells were cultured at 5% CO₂ and 37° C. for 24 to 48 h.

(7) Type IV Collagen Trimerization Assay

The type IV collagen α3-SmBiT (pFC36K SmBiT vector), α4-FLAG (pEB multi Hyg vector), and α5-LgBiT (pFC34K LgBiT vector) were transiently expressed in HEK 293T cells by lipofection of (6). Twenty four hours after the transfection, the cells were re-seeded at 3×10⁴ on a 96-well plate (White flat bottom, Thermo). Twelve hours after the re-seeding, the culture medium was changed to a phenol red-free culture medium (DMEM with 10% FBS and 200 mM 2P-AsA (ascorbyl 2-phosphate)). Twenty four hours after the final medium change, the culture supernatant was transferred to a new well, and a fresh culture medium (DMEM with 10% FBS and 200 mM 2P-AsA) was added to the cell-containing well. A luminescence reagent NanoGlo Live Cell Assay System (Promega) was added to each well. Then, after the mixture was allowed to stand in the dark for 10 min, luminescence was measured in accordance with instructions attached to the luminescence reagent. The luminescence was measured with a GloMax Navigator (Promega).

In addition, for comparison, α3-SmBiT (pFC36K SmBiT vector) alone, α5-LgBiT (pFC35K LgBiT vector) alone, or α3-SmBiT (pFC36K SmBiT vector) and α5-LgBiT (pFC34K LgBiT vector) were transiently expressed in H293T cells by lipofection. The respective cells prepared were likewise cultured and the luminescence was then measured.

Results

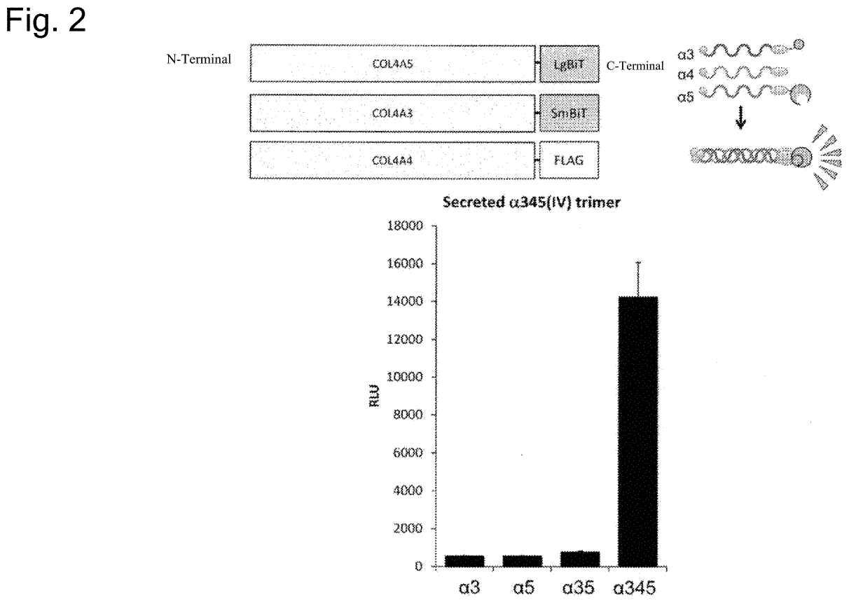

A luciferase reaction-mediated luminescence emission was specifically detected in a culture supernatant from the cells expressing type IV collagen α3-SmBiT, α4-FLAG, and α5-LgBiT (FIG. 2). By contrast, almost no luminescence emission was detected in a culture supernatant from the cells expressing α3-SmBiT alone, α5-LgBiT alone, or α3-SmBiT and α5-LgBiT. That is, as a result of intracellular type IV collagen trimerization, an extracellularly secreted type IV collagen trimer was able to be detected. These results demonstrate that use of this evaluation system makes it possible to evaluate a potential of type IV collage trimerization.

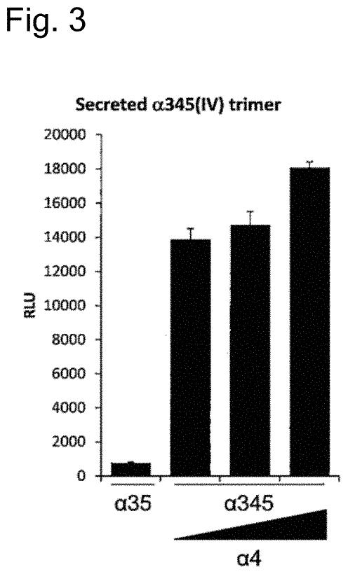

In addition, the transfection amount of the α4-FLAG (pEB multi Hyg vector) plasmid was varied, so that the level of expression of type IV collagen α4 chain was changed. As a result, it was observed that the level of type IV collagen trimer in the cell culture supernatant was increased in an α4 chain level-dependent manner (FIG. 3).

### Example 2: Effects of Domain-Deleted α5 Chains

Instead of the nucleic acid encoding wild-type α5 chain, a nucleic acid (nucleotides 4399 to 5073 of SEQ ID NO: 5) encoding NC1 domain of the wild-type α5 chain or a nucleic acid (nucleotides 124 to 4398 of SEQ ID NO: 5) encoding COL domain of the wild-type α5 chain was used to evaluate a potential of trimerization like Example 1. In this way, effects of the domain-deleted α5 chains were investigated.

The wild-type type IV collagen α5 chain includes, from the N-terminal side, a signal sequence, COL domain, and NC1 domain. Thus, the above nucleic acids were used to generate fusion proteins having a domain-deleted α5 chain. A fusion protein having a split luciferase fragment at the C terminal of the NC1 domain is denoted by 4COL; and a fusion protein having a split luciferase fragment at the C terminal of the COL domain is denoted by 4NC1.

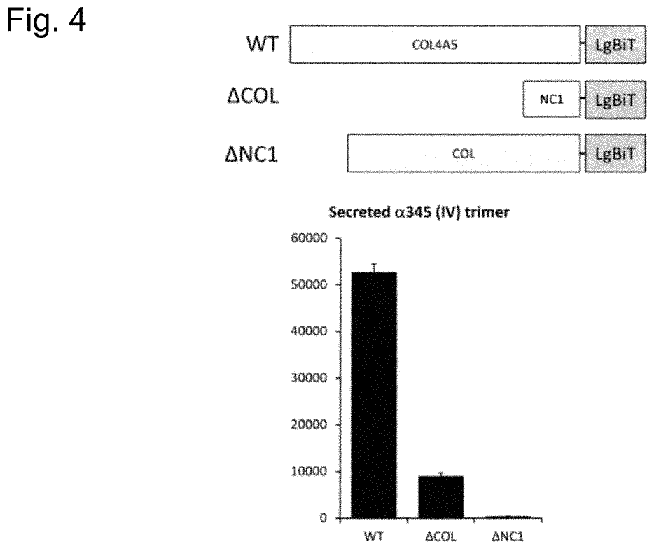

The results are shown in FIG. 4. When a fusion protein containing the wild-type type-IV collagen α5 chain, together with the α3 and α4 chains, was expressed, a trimer was observed in the culture supernatant (secreted product). By contrast, when each domain-deleted α5 chain, together with the α3 and α4 chains, was expressed, no trimer was observed. Thus, it has been demonstrated that each α5 chain domain deletion causes a potential of trimerization to decrease.

### Example 3: Trimer is Undetected when Single-Expression Cells are Co-Cultured

In Example 1, the type IV collagen α3-SmBiT, α4-FLAG, and α5-LgBiT were co-expressed in a single cell of HEK293T cells, and a secreted type IV collagen trimer was detected.

In this Example, for comparison, α3-SmBiT single-expression cells, α4-FLAG single-expression cells, and α5-LgBiT single-expression cells were prepared. These three types of cells were co-cultured and the type IV collagen trimerization was evaluated. The α3-SmBiT single-expression cells, the α4-FLAG single-expression cells, and the α5-LgBiT single-expression cells were prepared such that type IV collagen α3-SmBiT (pFC36K SmBiT vector), α4-FLAG (pEB multi Hyg vector), or α5-LgBiT (pFC34K LgBiT vector) was transiently expressed in HEK293T cells by lipofection of (6) in Example 1. The type IV collagen trimerization was assayed like Example 1.

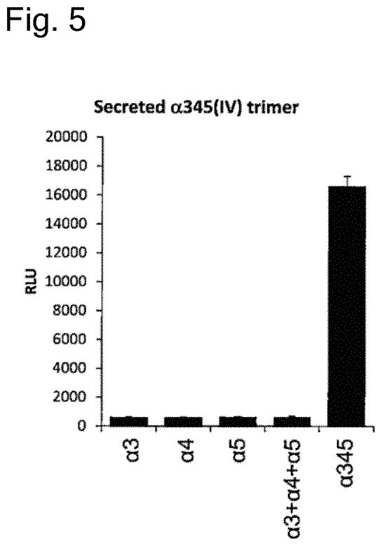

The results are shown in FIG. 5. When the α3-SmBiT, the α4-FLAG, and the α5-LgBiT were co-expressed in a single cell, a trimer in the culture supernatant (secreted product) was observed. By contrast, when the α3-SmBiT single-expression cells, the α4-FLAG single-expression cells, and the α5-LgBiT single-expression cells were co-cultured, almost no trimer was detected. These results are consistent with a type IV collagen intracellular regulation mechanism where type IV collagen that does not form a trimer inside a cell is not secreted extracellularly.

### Example 4: To Evaluate Potential of Trimerization with Each α5 Chain Mutant

Instead of the nucleic acid encoding the wild-type α5 chain, a nucleic acid encoding an α5 chain mutant containing each point mutation was used to prepare cells co-expressing the α3-SmBiT, the α4-FLAG, and the mutant α5-LgBiT by substantially the same procedure as of Example 1.

The point mutations of type IV collagen α5(VI) chain as examined in this Example include G129E, G153D, G227S, G325R, G426R, G475S, G521D, G573D, G594D, G594S, G624D, G650D, L664N, G675S, G796D, G796R, G869R, G911E, S916G, G953V, G1030S, G1107R, G1143D, G1170S, G1220D, G1241C, G1241V, G1244D, G1448R, P1517T, C1567R, R1569Q, M1607I, L1649R, and R1683Q. Here, each point mutation was denoted by “X₁nX₂”. Then, n indicates the position of an amino acid in the α5(IV) chain and agrees with the amino acid number of SEQ ID NO: 6. X₁ indicates an amino acid in a wild-type sequence; and X₂ indicates an amino acid in a mutated sequence. X₁ and X₂ are each expressed by amino acid one letter code well-known to those skilled in the art.

G869R among the above is the most frequently found mutation in patients with Alport syndrome. In addition, G1244D has been reported as a gene aberration in Alport syndrome. The frequency, however, is not understood. This mutation was found in patient A who developed a symptom of Alport syndrome.

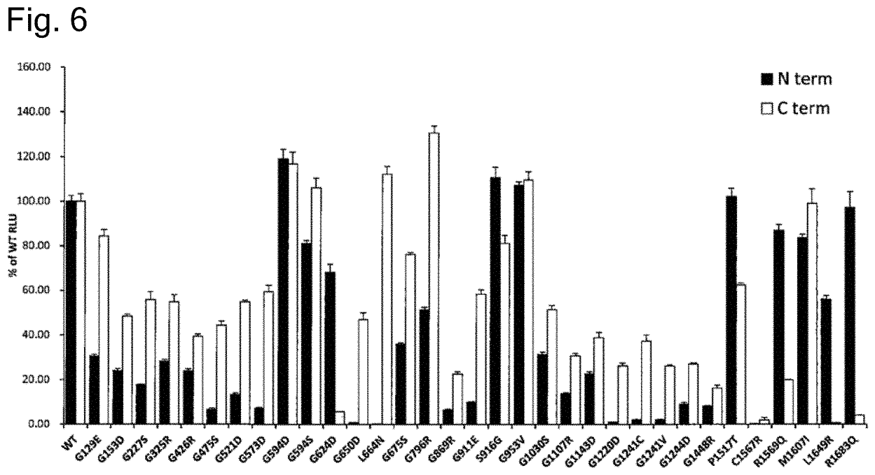

The results are shown in FIG. 6. The level of trimer detected in a culture supernatant when a mutant α5-LgBiT containing an α5 chain having a G869R mutation, which has been most frequently found in patients with Alport syndrome, was used was markedly lower than when the wild-type α5-LgBiT was used. In addition, the level of trimer detected in a culture supernatant with respect to a mutant α5-LgBiT containing an α5 chain having a G1244D mutation was markedly lower than when the wild-type α5-LgBiT was used. The symptom of the G1244D mutation does not contradict that of patient A. Further, the other mutations were also able to be determined such that some mutations elicited substantially the same level of trimerization as of the wild-type α5-LgBiT and others elicited a lower level of trimerization than that of the wild-type α5-LgBiT. These results demonstrate that use of this evaluation system makes it possible to quantitatively evaluate a potential of trimerization with any type IV collagen mutant.

### Example 5: α3 Chain and α5 Chain, N-Terminal Side of which was Fused to Split Luciferase Fragment)

In Example 1, established was the evaluation system where each fusion protein, in which the C-terminal side of α3 or α5 chain was fused to a split luciferase fragment, was expressed.

In this Example, established was an evaluation system where each fusion protein, in which the N-terminal side of α3 or α5 chain was fused to a split luciferase fragment, was expressed.

A DNA plasmid containing a nucleic acid encoding a fusion protein (SmBiT-α3) having SmBiT on the N-terminal side of α3 chain was constructed by subcloning a signal sequence-deleted COL4A3 (nucleotides 127 to 5013 of SEQ ID NO: 1) into a pFN36K SmBiT vector (Promega). The constructed DNA plasmid includes, in sequence from the 5′ end, Igκ leader sequence (SEQ ID NO: 11), a nucleic acid encoding SmBiT (SEQ ID NO: 7), and a nucleic acid linked to a signal sequence-deleted COL4A3 (nucleotides 127 to 5013 of SEQ ID NO: 1), and is an SmBiT-α3-expressing vector.

A DNA plasmid containing a nucleic acid encoding a fusion protein (LgBiT-α5) having LgBiT on the N-terminal side of α5 chain was constructed by subcloning a signal sequence-deleted COL4A5 (nucleotides 124 to 5073 of SEQ ID NO: 5) into a pFN33K LgBiT vector (Promega). The constructed DNA plasmid includes, in sequence from the 5′ end, Igκ leader sequence (SEQ ID NO: 11), a nucleic acid encoding LgBiT (SEQ ID NO: 9), and a nucleic acid linked to a signal sequence-deleted COL4A5 (nucleotides 124 to 5073 of SEQ ID NO: 5), and is an LgBiT-α5-expressing vector.

The same experiment as of Example 1 was repeated except that the DNA plasmids for the α3 chain and the α5 chain were constructed as above. The following Table 2 shows combinations of each gene and a plasmid containing the gene.

TABLE 2

| | | From which | Fusion protein |
|---|---|---|---|
| | | vendor the | that the expression |
| Gene | | vector was | vector after the |
| name | Vector | obtained | subcloning encodes |
| COL4A3 | pFN35K SmBiT | Promega | SmBiT-α3 |
| COL4A5 | pFN33K LgBiT | Promega | LgBiT-α5 |
| COL4A4 | pEB Multi Hyg | Wako | α4-FLAG |

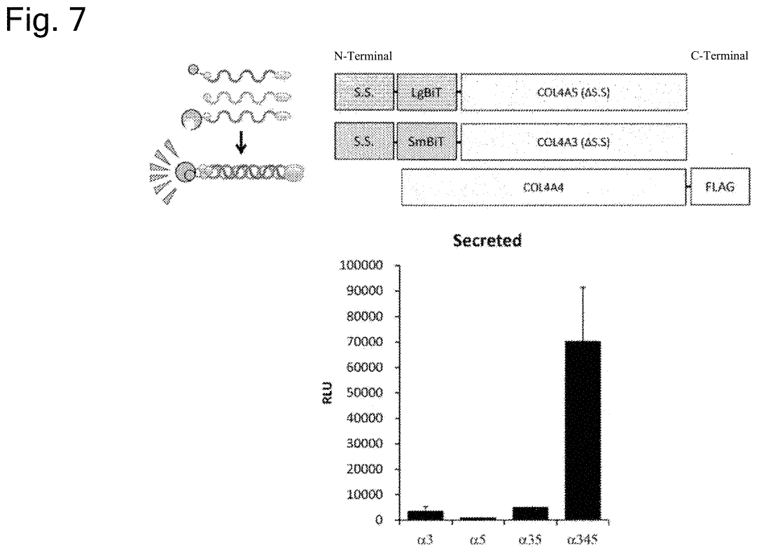

The results are shown in FIG. 7. In the evaluation system of this Example, a trimer of type IV collagen was detected in the manner similar to the evaluation system of Example 1. This result demonstrates that each split luciferase fragment may be fused on any of the N-terminal side and the C-terminal side of α3 chain or α5 chain.

### INDUSTRIAL APPLICABILITY

The method for evaluating a potential of type IV collagen trimerization according to the present invention allows for evaluation using, for instance, a 96-well plate. This enables a compound, which promotes and stabilizes type IV collagen trimerization, to be searched through high-throughput screening. The compound, which promotes and stabilizes type IV collagen trimerization, may be utilized as a therapeutic drug for Alport syndrome and can be a powerful tool in drug development.

1. A method for evaluating a potential of type IV collagen trimerization, comprising
(1) culturing cells co-expressing the following fusion proteins (a) to (c):
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment,
(2) adding a luminescent substrate to a culture product of (1) and carrying out an incubation thereof, and
(3) evaluating a potential of type IV collagen trimerization in accordance with a luminescence emission intensity.

2. The method according to claim 1, wherein the cells co-expressing the fusion proteins (a) to (c) are obtained by transfecting a cell with:
(a′) an expression vector comprising a gene encoding a fusion protein comprising a wild-type or mutant type IV collagen α3(IV) chain and one of split luciferase fragments;
(b′) an expression vector comprising a gene encoding a fusion protein comprising a wild-type or mutant type IV collagen α4(IV) chain and a peptide tag; and
(c′) an expression vector comprising a gene encoding a fusion protein comprising a wild-type or mutant type IV collagen α5(IV) chain and the other split luciferase fragment.

3. The method according to claim 1 or 2, wherein the fusion proteins (a) to (c) are:
(a) a fusion protein comprising one of split luciferase fragments on a C-terminal side of a wild-type or mutant type IV collagen α3(IV) chain and;
(b) a fusion protein comprising a peptide tag on a C-terminal side of a wild-type or mutant type IV collagen α4(IV) chain; and
(c) a fusion protein comprising the other split luciferase fragment on a C-terminal side of a wild-type or mutant type IV collagen α5(IV) chain.

4. The method according to claim 1 or 2, wherein the fusion proteins (a) to (c) are:
(a) a fusion protein comprising one of split luciferase fragments on an N-terminal side of a wild-type or mutant type IV collagen α3(IV) chain and;
(b) a fusion protein comprising a peptide tag on a C-terminal side of a wild-type or mutant type IV collagen α4(IV) chain; and
(c) a fusion protein comprising the other split luciferase fragment on an N-terminal side of a wild-type or mutant type IV collagen α5(IV) chain.

5. The method according to claim 1, wherein the peptide tag is FLAG tag (SEQ ID NO: 12) or 3×FLAG tag (SEQ ID NO: 13).

6. The method according to claim 1,
wherein in step (1), a first portion of the cells are cultured in the presence of a candidate compound and a second portion of the cells are cultured in the absence of the candidate compound the method further comprising:
(4) comparing a luminescence emission intensity of the culture product cultured in the presence of the candidate compound with a luminescence emission intensity of the culture product cultured in the absence of the candidate compound, and
(5) identifying the candidate compound as a compound that promotes a potential of type IV collage trimerization when the luminescence emission intensity of the culture product cultured in the presence of the candidate compound is higher than the luminescence emission intensity of the culture product cultured in the absence of the candidate compound.

7. The method according to claim 1,
wherein in step (1), the cells are cultured in the presence of each of a serially diluted candidate compound, the method further comprising
(4) evaluating, based on a luminescence emission intensity according to a concentration of the candidate compound, concentration dependency of the candidate compound with regard to promoting a potential of type IV collagen trimerization.

8. The method according to claim 1,
wherein in step (1) the cells are cultured in the presence of each of a plurality of candidate compounds, the method further comprising:
(4) measuring a luminescence emission intensity in the presence of each candidate compound to determine a candidate compound exhibiting a higher luminescence emission intensity as a compound with a higher effect of promoting a potential of type IV collagen trimerization.

9. A kit for evaluating a potential of type IV collagen trimerization, screening for a compound that promotes a potential of type IV collagen trimerization, or evaluating a therapeutic drug for Alport syndrome, the kit comprising:
(a′) an expression vector comprising a gene encoding a fusion protein comprising a wild-type or mutant type IV collagen α3(IV) chain and one of split luciferase fragments;
(b′) an expression vector comprising a gene encoding a fusion protein comprising a wild-type or mutant type IV collagen α4(IV) chain and a peptide tag; and
(c′) an expression vector comprising a gene encoding a fusion protein comprising a wild-type or mutant type IV collagen α5(IV) chain and the other split luciferase fragment.

10. A kit for evaluating a potential of type IV collagen trimerization, screening for a compound that promotes a potential of type IV collagen trimerization, or evaluating a therapeutic drug for Alport syndrome, the kit comprising cells co-expressing:
(a) a fusion protein comprising a wild-type or mutant type IV collagen α3(IV) chain and one of split luciferase fragments;
(b) a fusion protein comprising a wild-type or mutant type IV collagen α4(IV) chain and a peptide tag; and
(c) a fusion protein comprising a wild-type or mutant type IV collagen α5(IV) chain and the other split luciferase fragment.
